(19) United States
(12) Patent Application Publication
(10) Pub. No.: US 2022/0296597 A1
(43) Pub. Date: Sep. 22, 2022

(54) USE OF A NEUTROPHIL ELASTASE INHIBITOR IN LUNG DISEASE

(71) Applicant: pH Pharma Co., Ltd., Gyeonggi-do (KR)

(72) Inventors: Sanjeev SATYAL, San Carlos, CA (US); Hoyoung HUH, Portola Valley, CA (US)

(73) Assignee: pH Pharma Co., Ltd., Gyeonggi-do (KR)

(21) Appl. No.: 17/636,788

(22) PCT Filed: Aug. 21, 2020
(86) PCT No.: PCT/US20/47528

§ 371 Date: Feb. 18, 2022

Related U.S. Application Data
(60) Provisional application No. 62/890,774, filed on Aug. 23, 2019.

(51) Int. Cl. A61K 31/513; A61P 11/00

(52) U.S. Cl. CPC A61K 31/513; A61P 11/00

(57) ABSTRACT

The invention relates to methods for treating chronic lung disease, in particular, alpha-1 antitrypsin deficiency or emphysema resulting from alpha-1 antitrypsin deficiency, with a neutrophil elastase inhibitor. The invention further relates to pharmaceutical compositions comprising a neutrophil elastase inhibitor.

### CROSS-REFERENCE TO RELATED APPLICATIONS

The present application claims priority to U.S. Provisional Patent Application No. 62/890,774, filed on Aug. 23, 2019, the contents of which is herein incorporated by reference in its entirety.

### TECHNICAL FIELD

The invention relates to methods for chronic lung disease, in particular, treating alpha-1 antitrypsin deficiency or emphysema resulting from alpha-1 antitrypsin deficiency, with a neutrophil elastase inhibitor. The invention further relates to pharmaceutical compositions comprising a neutrophil elastase inhibitor.

### BACKGROUND OF THE INVENTION

Alpha-1 antitrypsin deficiency (AATD) is an autosomal recessive hereditary disorder associated with emphysema and, less frequently, with liver cirrhosis (Crystal, R. G., The Lancet, 2017, Vol. 5, http://dx.doi.org/10.1016/52213-2600(16)30434-9). It is caused by mutations in the SERPINA1 gene, which encodes the protease inhibitor alpha-1 antitrypsin (AAT or A1AT). Alpha-1 antitrypsin A is a protease inhibitor. It is also known as alpha1-proteinase inhibitor (A1PI) or alpha1-antiproteinase (A1AP) because it inhibits various proteases in addition to trypsin including neutrophil elastase (Gettins, P. G., Chemical Reviews, 2002, 102(12):4751-804). Absence or deficiency of AAT leads to an imbalance between elastase and anti-elastase activity, which results in progressive, irreversible destruction of lung tissue, and ultimately the development of chronic obstructive pulmonary disease (COPD) with early-onset emphysema (Rahaghi, F. F. and Miravitlles, M., Respiratory Res., 2017, 18:105). AATD is a rare, slowly progressive disease, which can take decades to manifest clinically (Wewers, M. D., Crystal, R. G., COPD, 2013, 10(Suppl. 1):64-7). AATD also causes liver fibrosis in certain patients due to the inactive, mutant AAT accumulating in the liver. This condition also occurs in children. These conditions often remain undiagnosed until serious pathology occurs because liver injury and fibrosis are not accurately detected by available routine liver screenings (Teckman, J. H., et al., “Alpha-1 Antitrypsin Deficiency”, in Pathophysiology of Alpha-1 Antitrypsin Deficiency Liver Disease, 2017, Vol. 1639, pp. 1-).

The most frequent disease-associated SERPINA1 mutations are referred to as the “S” and “Z” alleles, with the “Z” mutation leading to the most severe disease symptoms and is the most heavily studied subpopulation (Greene et al., Thorax, 2015, 70:939-945). Prevalence of these mutations in Europe is estimated to be between 1 in 2,000 and 1 in 5,000 individuals. S and Z alleles lead to production of misfolded AAT, resulting in reduced secretion of AAT from hepatocytes into circulation (Greene et al., 2016). Patients with AATD show low or undetectable levels of circulating AAT. Inherited ZZ AATD accounts for approximately 1% of COPD cases (Rahaghi et al., COPD: J. Chronic Obstructive Pulm. Dis., 2012, 9(4):352-8) and is the fourth most common reason for lung transplantation (Yusen et al., J. Heart Lung Transplant, 2013, 32(10):965-7). Diagnosis of AATD often first entails the determination of low serum AAT level (considered to be <11 μM (0.5 g/L) followed by phenotyping (Miravitlles et al., Eur. Resp. J., 2017, 50:1700610). Despite the availability of genetic testing, AATD is often underdiagnosed, owing in part to the lack of nationwide screening programs and awareness within Europe (Horvath et al., ERJ Open Res., 2019, 5(1):00171-2018; Miravitlles et al., 2017). Of the over 119,000 individuals estimated to carry the high risk Pi*ZZ genotype within Europe (Blanco et al., J. COPD., 2017, 12:561-569 and 1683-1694), physicians estimate that only 15% of these individuals have been accurately diagnosed with AATD. For example, within Ireland specifically, it is estimated that only 300 of more than 2,200 individuals with severe AATD have been diagnosed (Carroll, T. P., et al., Respiratory Research, 2011, 12:91).

The current standard treatment for AATD is augmentation therapy, also known as replacement therapy. Augmentation therapy is the use of AAT protein purified from the blood plasma of healthy human donors to increase the patient's AAT levels. Commercially available AAT preparations include Prolastin and Prolastin-C® (Grifols, Barcelona, Spain), Alfalastin (LFB, Courtaboeuf Cedex, France), Aralast® NP (Baxalta US, Inc., Lexington, Mass.), Zemaira® and Respreeza (CSL Behring, King of Prussia, Pa.), and Glassia® (Baxalta US, Inc., Lexington, Mass.). All of these medications carry a risk of transmitting blood-borne infectious agents including viruses such as hepatitis and HIV, and theoretically, the Creutzfeldt-Jakob disease agent (a prion), despite manufacturing steps designed to minimize the risk of transmission of these vectors. These medications must be administered by intravenous infusion, typically once a week, a process that is at best uncomfortable with a serious impact on the patient's quality of life. Clearly, improved therapies for AATD and related conditions are needed.

### SUMMARY OF THE INVENTION

In one aspect, the invention provides a method of treating chronic lung disease, comprising administering a therapeutically effective amount of (4S)-4-[4-cyano-2-(methylsulfonyl)phenyl]-3,6-dimethyl-2-oxo-1-[3-(trifluoromethyl)phenyl]-1,2,3,4-tetrahydropyrimidine-5-carbonitrile or a pharmaceutically acceptable salt, polymorph, solvate, or solvates of the salts thereof to a patient in need of treatment, wherein the therapeutically effective amount comprises a dosage of 1 mg, 2 mg, 5 mg, 10 mg, 20 mg or 40 mg once a day, and wherein the chronic lung disease is selected from the group consisting of alpha-1 antitrypsin deficiency or emphysema resulting from alpha-1 antitrypsin deficiency. In one embodiment, the chronic lung disease comprises alpha-1 antitrypsin deficiency. In another embodiment, the chronic lung disease comprises emphysema resulting from alpha-1 antitrypsin deficiency. In one embodiment, the method further comprises administering one or more additional therapies. In another embodiment, the additional therapy comprises augmentation therapy with human alpha-1 antitrypsin. In another embodiment, the additional therapy comprises a therapeutic agent when administered to a patient by itself treats or ameliorates alpha-1 antitrypsin deficiency emphysema resulting from alpha-1 antitrypsin deficiency. In a further embodiment, the therapeutic agent is an alpha-1 antitrypsin modulator, gene therapy, RNA-based therapy, a leukocyte elastase inhibitor, or recombinant AAT.

In another aspect, the invention provides a pharmaceutical composition for the treatment of alpha-1 antitrypsin deficiency or emphysema resulting from alpha-1 antitrypsin deficiency comprising (4S)-4-[4-cyano-2-(methylsulfonyl)phenyl]-3,6-dimethyl-2-oxo-1-[3-(trifluoromethyl)phenyl]-1,2,3,4-tetrahydropyrimidine-5-carbonitrile or a pharmaceutically acceptable salt, polymorph, solvate, or solvates of the salts thereof and a pharmaceutically acceptable carrier. In another embodiment, the pharmaceutical composition is formulated as a tablet. In a further embodiment, the tablet comprises one or more diluents, disintegrants, surfactants or lubricants. In another embodiment, the pharmaceutical composition comprises 1 mg, 2 mg, 5 mg, 10 mg, 20 mg or 40 mg of (4S)-4-[4-cyano-2-(methylsulfonyl)phenyl]-3,6-dimethyl-2-oxo-1-[3-(trifluoromethyl)phenyl]-1,2,3,4-tetrahydropyrimidine-5-carbonitrile or a pharmaceutically acceptable salt, polymorph, solvate, or solvates of the salts thereof.

In another aspect, the invention provides a method of treating alpha-1 antitrypsin deficiency or emphysema resulting from alpha-1 antitrypsin deficiency in a patient in need of such treatment comprising administering to said patient a therapeutically effective amount of a pharmaceutical composition comprising (4S)-4-[4-cyano-2-(methylsulfonyl)phenyl]-3,6-dimethyl-2-oxo-1-[3-(trifluoromethyl)phenyl]-1,2,3,4-tetrahydropyrimidine-5-carbonitrile or a pharmaceutically acceptable salt, polymorph, solvate, or solvates of the salts thereof and a pharmaceutically acceptable carrier. In one embodiment, the pharmaceutical composition comprises 1 mg, 2 mg, 5 mg, 10 mg, 20 mg or 40 mg of (4S)-4-[4-cyano-2-(methylsulfonyl)phenyl]-3,6-dimethyl-2-oxo-1-[3-(trifluoromethyl)phenyl]-1,2,3,4-tetrahydropyrimidine-5-carbonitrile or a pharmaceutically acceptable salt, polymorph, solvate, or solvates of the salts thereof.

In another aspect, the invention provides a compound (4S)-4-[4-cyano-2-(methylsulfonyl)phenyl]-3,6-dimethyl-2-oxo-1-[3-(trifluoromethyl)phenyl]-1,2,3,4-tetrahydropyrimidine-5-carbonitrile or a pharmaceutically acceptable salt, polymorph, solvate, or solvates of the salts thereof for use for the therapeutic treatment of chronic lung disease, wherein the (4S)-4-[4-cyano-2-(methylsulfonyl)phenyl]-3,6-dimethyl-2-oxo-1-[3-(trifluoromethyl)phenyl]-1,2,3,4-tetrahydropyrimidine-5-carbonitrile or a pharmaceutically acceptable salt, polymorph, solvate, or solvates of the salts thereof is administered in a dosage of 1 mg, 2 mg, 5 mg, 10 mg, 20 mg or 40 mg once a day, wherein the chronic lung disease is selected from the group consisting of alpha-1 antitrypsin deficiency or emphysema resulting from alpha-1 antitrypsin deficiency. In one embodiment, the chronic lung disease is alpha-1 antitrypsin deficiency. In another embodiment, the chronic lung disease is emphysema resulting from alpha-1 antitrypsin deficiency. In one embodiment, the method further comprises administering one or more additional therapies. In another embodiment, the additional therapy comprises augmentation therapy with human alpha-1 antitrypsin. In another embodiment, the additional therapy comprises a therapeutic agent when administered to a patient by itself treats or ameliorates alpha-1 antitrypsin deficiency emphysema resulting from alpha-1 antitrypsin deficiency. In a further embodiment, the therapeutic agent is an alpha-1 antitrypsin modulator, gene therapy, RNA-based therapy, a leukocyte elastase inhibitor, or recombinant AAT.

Other objects of the invention may be apparent to one skilled in the art upon reading the following specification and claims.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows the total synthesis of (4S)-4-[4-cyano-2-(methylsulfonyl)phenyl]-3,6-dimethyl-2-oxo-1-[3-(trifluoromethyl)phenyl]-1,2,3,4-tetrahydropyrimidine-5-carbonitrile as described in U.S. Pat. No. 8,288,402 (Von Nussbaum). The reaction scheme is as follows: the reaction sequence from a compound of formula (II) through the compounds of formula (III), (IV) and (V) to a compound of formula (VI) in Scheme 6 and Examples 1A, 2A Method B, and 3A Method B and 4A Method B of the Von Nussbaum patent; the reaction sequence from a compound of formula (VI) through a compound of formula (IX) to a compound of formula (X) in Scheme 1 and Examples 3 and 4 of the Von Nussbaum patent; and the reaction sequence from a compound of formula (X) through the compounds of formulas (XI) and (XII) to a compound of (XIII) in Scheme 2 and Examples 5A, 5 and 6 of the Von Nussbaum patent. The synthesis of the compound of formula (I) (Compound 1 herein) is described in Example 33 Method B of the Von Nussbaum patent.

### DETAILED DESCRIPTION OF THE INVENTION

This application is not limited to particular methodologies or the specific compositions described, because the scope of the present application will be limited only by the appended claims and their equivalents. It is also to be understood that the terminology used herein is for the purpose of describing particular embodiments only, and is not intended to be limiting.

Unless defined otherwise, all technical and scientific terms used herein have the same meaning as commonly understood by one of ordinary skill in the art to which this application belongs. It must be noted that as used herein and in the appended claims, the singular forms “a”, “and”, and “the” include plural referents unless the context clearly dictates otherwise.

Reference will now be made in detail to certain preferred methods of treatment, compounds and methods of administering these compounds. The invention is not limited to those preferred compounds and methods, but rather is defined by the claim(s) issuing herefrom.

### INTRODUCTION

The present invention provides a method for treating alpha-1 antitrypsin deficiency (AATD) and emphysema resulting from AATD using Compound 1, (4S)-4-[4-cyano-2-(methylsulfonyl)phenyl]-3,6-dimethyl-2-oxo-1-[3-(trifluoromethyl)phenyl]-1,2,3,4-tetrahydropyrimidine-5-carbonitrile or a pharmaceutically acceptable salt, polymorph, solvate, or solvates of the salts thereof. The present invention also provides pharmaceutical compositions of Compound 1 suitable for use in the treatment of AATD and emphysema resulting from AATD.

Compound 1 has previously been disclosed as a potent neutrophil elastase (NE) inhibitor (Nagelschmitz, J., et al., European Respiratory J., 2014, 44, Suppl. 58, Abstract no. 3416). It is approximately 100 times more selective for human NE (Kᵢ [M]=8.0×10⁻¹¹) than for murine neutrophil elastase (Kᵢ [M]=6.0×10⁻⁹) (Von Nussbaum, F., et al., ChemMedChem., 2015, 10:1163-1173). Human neutrophil elastase (hNE) is a very active serine protease secreted by neutrophils during inflammation. It is also known as human leukocyte elastase (HLE, EC 3.4.21.37). This proteolytic enzyme is found in the azurophilic granules of polymorphonuclear leukocytes (PMN leukocytes). The intracellular elastase plays an important role in defense against pathogens by breaking down foreign particles which are taken up through phagocytosis (Nagelschmitz, 2014). The highly active proteolytic enzyme is able to break down a multitude of connective tissue proteins, such elastin, collagen and fibronectin. Elastin occurs in high concentrations in all tissue types exhibiting high elasticity, such as in the lungs and in arteries. NE is also an important modulator of inflammatory processes. An excess of hNE activity has been implicated in the pathogenesis of inflammatory pulmonary diseases like bronchiectasis, COPD and pulmonary arterial hypertension.

Compound 1 has been disclosed as a treatment for various pulmonary diseases and for the treatment of chronic wounds in a number of patents and applications (U.S. Pat. Nos. 8,288,402; 8,889,700; 9,174,997; PCT Publication WO 2017/081044), the disclosures of which are herein incorporated by reference). In particular, U.S. Pat. No. 8,288,402 discloses the use of Compound 1 in the treatment of pulmonary arterial hypertension and acute lung failure.

The safety and tolerability of Compound 1, also known as BAY 85-8501, has been evaluated in several human clinical trials. Four clinical studies, including two Phase 1, single-dose studies in healthy subjects, a Phase 1, multiple-dose study in healthy subjects, and a Phase 2a, multiple-dose study in subjects with non-cystic fibrosis bronchiectasis (nCF BE), have assessed the safety, pharmacokinetics (PK), and pharmacodynamics (PD) of Compound 1 administered as an oral solution and/or an immediate-release (IR) tablet. In healthy subjects participating in the three Phase 1 studies, single and repeated Compound 1 treatments administered at doses up to 1 mg for up to 14 days were safe and well tolerated. Adverse events (AEs) reported in the Phase 1 studies were generally mild and unrelated to study treatment, and no serious AEs (SAEs) were reported. No safety signals for study drug-induced laboratory or electrocardiogram (ECG) abnormalities were observed. (See: Nagleschmitz, J., et al., European Respiratory J., 2014, 44:3416; Nagelschmitz, J., et al., European Respiratory J., 2014, 44:P1511).

A multi-center, Phase 2a, randomized, double-blind, placebo-controlled study in subjects with non-CF BE was conducted using a 28-day oral administration of Compound 1 (www.clinicaltrials.gov; Identifier: NCT01818544). Ninety-four patients (mean age, 66 years, 53% male) were randomized to treatment with 45 patients receiving a 1.0 mg oral dose of Compound 1 administered as an IR tablet. The drug was generally safe and well tolerated over 28 days. Safety results for subjects receiving Compound 1 and placebo were generally similar. AEs were generally mild or moderate in severity, unrelated to study treatment, and not different between study drug and placebo. The incidence of SAEs and withdrawals of study treatment due to AEs was low and no SAEs were attributed by the investigator to the drug. No safety signals for study drug-induced laboratory parameter or ECG effects were observed. (See: Watz, H., et al., European Respiratory J., 2016, 48:PA4088).

### Chemical Description

Compound 1, (4S)-4-[4-cyano-2-(methylsulfonyl)phenyl]-3,6-dimethyl-2-oxo-1-[3-(trifluoromethyl)phenyl]-1,2,3,4-tetrahydropyrimidine-5-carbonitrile, has the following chemical structure:

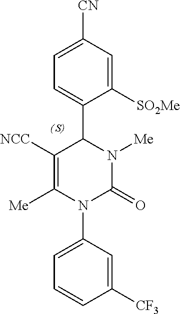

Alternatively, Compound 1 may be named (4S)-4-[4-cyano-2-(methylsulfonyl)phenyl]-1,2,3,4-tetrahydro-3,6-dimethyl-2-oxo-1-[3-(trifluoromethyl)phenyl]-5-pyrimidinecarbonitrile. Compound 1 is commonly known in the literature as BAY 85-8501. It is understood that any of these designations for Compound 1 may be interchangeably used and have the same meaning.

Compound 1 and its salts, polymorphs, solvates, or solvates of salts may exist in various stereoisomeric forms, i.e. in the form of configurational isomers or, if appropriate, also as conformational isomers (enantiomers and/or diastereomers, including atropisomers). Compound 1 therefore also refers to the enantiomers and diastereomers and to their respective mixtures. The stereoisomerically pure constituents can be isolated in a known manner from such mixtures of enantiomers and/or diastereomers. Compound 1 also encompasses any possible tautomeric forms.

Compound 1 may exist in multiple physical forms, including but not limited to, multiple crystalline forms, non-crystalline amorphous forms, and polymorphs. In general, all physical forms are equivalent for the uses contemplated herein and are intended to be within the scope of the present disclosure. Polymorphism refers to the ability of a molecule to exist in two or more crystalline forms in which the molecules with a crystal lattice may differ in structural arrangement and/or conformation. Polymorphic structures have the same chemical composition, although their different lattice structures and/or conformations can result in different physical, chemical or pharmacological properties, such as solubility, stability, melting point, density and bioavailability. Amorphous forms do not have a defined crystal structure. All polymorphs and other physical forms of Compound 1 are equivalent for the uses contemplated herein and are intended to be within the scope of the present invention.

Salts which are preferred for the purposes of the present invention are physiologically acceptable salts of Compound 1. Also encompassed are salts which are themselves unsuitable for pharmaceutical uses but can be used, for example, for isolating or purifying the compounds according to the invention. Salts may exist in multiple physical forms, including but not limited to, multiple crystalline forms, non-crystalline amorphous forms, and polymorphs.

Physiologically acceptable salts of Compound 1 include acid addition salts of mineral acids, carboxylic acids and sulfonic acids, for example salts of hydrochloric acid, hydrobromic acid, sulfuric acid, phosphoric acid, methanesulfonic acid, ethanesulfonic acid, toluenesulfonic acid, benzenesulfonic acid, naphthalenedisulfonic acid, acetic acid, trifluoroacetic acid, propionic acid, lactic acid, tartaric acid, malic acid, citric acid, formic acid, fumaric acid, maleic acid and benzoic acid. Physiologically acceptable salts of Compound 1 also include salts of conventional bases such as, by way of example and preferably, alkali metal salts (for example sodium salts and potassium salts), alkaline earth metal salts (for example calcium salts and magnesium salts) and ammonium salts derived from ammonia or organic amines having 1 to 16 carbon atoms, such as, by way of example and preferably, ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine and N-methylpiperidine.

Solvates refers for the purposes of the invention to those forms of Compound 1 according to the invention which form, in the solid or liquid state, a complex by coordination with solvent molecules. Solvates may exist in multiple physical forms, including but not limited to, multiple crystalline forms, non-crystalline amorphous forms, and polymorphs. Solvates may also form with the pharmaceutically acceptable salts of Compound 1. Hydrates are a specific form of solvates in which the coordination takes place with water. Various organic solvents may form solvates with Compound 1, including but not limited to, 1,4-dioxane, 1-propanol, 1-butanol, 1,2-dimethoxyethane, 2-ethoxyethanol, 2-methoxyethanol, 2-methyl-1-propanol, 2-methyl tetrahydrofuran, 3-methyl-1-butanol, acetic acid, acetone, acetonitrile, anisole, butyl acetate, chlorobenzene, cumene, dimethylsulfoxide, ethanol, ethyl acetate, ethyl ether, ethyl formate, ethylene glycol, formic acid, heptane, isobutyl acetate, isopropyl ether, isopropyl acetate, methanol, methyl acetate, methyl ethyl ketone, methylisobutyl ketone, N-methylpyrrolidone, tert-butanol, tert-butylmethyl ether, tetrahydrofuran and toluene.

### Chemical Synthesis

Compound 1, (4S)-4-[4-cyano-2-(methylsulfonyl)phenyl]-3,6-dimethyl-2-oxo-1-[3-(trifluoromethyl)phenyl]-1,2,3,4-tetrahydropyrimidine-5-carbonitrile, may be prepared as described by Von Nussbaum et al. (U.S. Pat. No. 8,288,402), which is herein incorporated by reference in its entirety. Alternatively, the method of Schirmer et al., as described in U.S. Published Application No. 2018/0072685, which is herein incorporated by reference in its entirety, may be used.

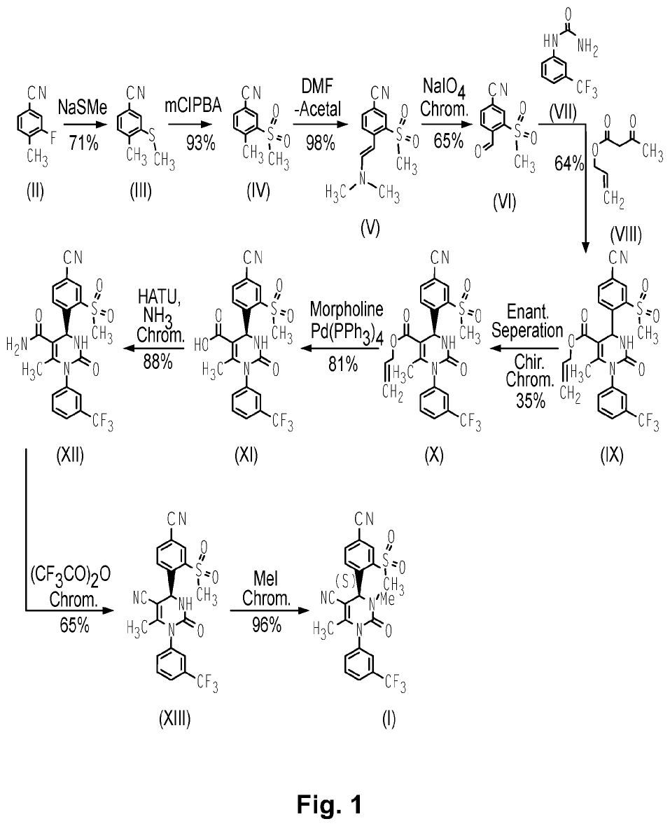

The method of Von Nussbaum et al. is depicted in U.S. Pat. No. 8,288,402. Starting from 3-fluoro-4-methylbenzonitrile, Compound 1 is produced in 10 steps with a total yield of 4.45% of theory. FIG. 1 shows in detail the intermediate steps in the synthesis. The final step is the N-methylation followed by column chromatography. The S-enantiomer is obtained by concentration of chromatography fractions as an amorphous solid. Further details of the synthesis may be found in Example 33 of the Von Nussbaum et al. patent.

Schrimer et al. provides an improved synthesis of Compound 1 as depicted in the schemes provided in U.S. Published Application No. 2018/0072685. The improved method is available in two variants, with method variant (A) furnishing Compound 1 in 8 steps (see Schemes 7, 2 and 3, of U.S. 2018/0072685) in more than 17% of theory overall yield without a chromatographic purification of intermediates. Method variant (B) (see Schemes 7, 4, 5 and 6, of U.S. 2018/0072685) furnishes Compound 1 in 9 steps, likewise without a chromatographic purification of intermediates, with the overall yield depending on the reaction management, as described in detail in U.S. 2018/0072685.

Compound 1 is a white to yellow solid, with a melting point of 232° C. It is considered neutral and does not readily form salts. Compound 1 is not hygroscopic under normal storage conditions. Compound 1 is practically insoluble in water, very slightly soluble in ethanol, and soluble in acetone.

### Pharmaceutical Compositions

Compositions containing (4S)-4-[4-cyano-2-(methylsulfonyl)phenyl]-3,6-dimethyl-2-oxo-1-[3-(trifluoromethyl)phenyl]-1,2,3,4-tetrahydropyrimidine-5-carbonitrile (Compound 1) or a pharmaceutically acceptable salt, polymorph, solvate, or solvates of salts thereof as the active ingredient may be advantageously used to treat chronic lung diseases. While it is possible for Compound 1 or a pharmaceutically acceptable salt, polymorph, solvate, or solvates of salts thereof to be administered alone, it is preferable to present it as a formulation. The compositions, or dosage forms, may be administered or applied singly, or in combination with other agents, including one or more diluents, disintegrants, surfactants or lubricants. The formulations may also deliver Compound 1 to a patient in combination with another pharmaceutically active agent.

The term “composition” as used herein is intended to encompass a product comprising specified ingredients in predetermined amounts or proportions, as well as any product which results, directly or indirectly, from combination of the specified ingredients in the specified amounts. This term in relation to pharmaceutical compositions is intended to encompass a product comprising one or more active ingredients, and an optional pharmaceutically acceptable carrier comprising inert ingredients, as well as any product which results, directly or indirectly, from combination, complexation or aggregation of any two or more of the ingredients, or from dissociation of one or more of the ingredients, or from other types of reactions or interactions of one or more of the ingredients. In general, pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases. Accordingly, the pharmaceutical compositions of the present invention encompass any composition made by admixing a compound of the present invention and a pharmaceutically acceptable carrier. Said compositions are prepared according to conventional mixing, granulating, or coating methods, respectively, and contain 0.1 to 75%, preferably 1 to 50%, of the active ingredient.

By “pharmaceutically acceptable” it is meant the carrier, diluent or excipient must be compatible with the other ingredients of the formulation and not deleterious to the recipient thereof. Pharmaceutical compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations.

Tablets contain the active ingredient in admixture with non-toxic pharmaceutically acceptable excipients which are suitable for the manufacture of tablets, including, but not limited to, diluents, disintegrants, surfactants and lubricants. These excipients may be, for example, inert diluents, such as calcium carbonate, sodium carbonate, lactose, calcium phosphate or sodium phosphate; granulating and disintegrating agents, for example, cornstarch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. A tablet may be made by compressing or molding the active ingredient optionally with one or more pharmaceutically acceptable ingredients. Compressed tablets may be prepared by compressing, in a suitable machine, the active ingredient in a free-flowing form such as a powder or granules, optionally mixed with a binder, lubricant, inert diluent, surface active, or dispensing agent. Molded tablets may be made by molding, in a suitable machine, a mixture of the powdered active ingredient and a suitable carrier moistened with an inert liquid diluent. Tablets may be prepared as described in the Examples below or as described in PCT Application WO 2017/081044 (May et al.), which is incorporated herein in its entirety.

Compositions for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil. In particular, a pharmaceutical composition of the present invention may comprise a liquid-filled capsule dosage form in which the active ingredient is in solution in certain combinations of liquid and semi-solid excipients.

Compositions for oral administration may also be formulated as aqueous suspensions containing the active ingredient in admixture with excipients suitable for the manufacture of aqueous suspensions. Oily suspensions may be formulated by suspending the active ingredient in a suitable oil. Oil-in-water emulsions may also be employed. Dispersible powders and granules suitable for preparation of an aqueous suspension by the addition of water provide the active ingredient in admixture with a dispersing or wetting agent, suspending agent and one or more preservatives. Oral suspensions of Compound 1 may be prepared as described in PCT Application WO 2017/081044 (May et al.).

The active ingredient of the present invention may be administered in an oral immediate release formulation as tablets, capsules, suspensions or emulsions for oral administration as described above.

Compound 1 may be administered by intravenous infusion. Solutions of Compound 1 suitable for intravenous administration may be prepared as described in PCT Published Application No. WO 2017/081044 (May et al.).

Suitable topical formulations and dosage forms include ointments, creams, gels, lotions, pastes, and the like, as described in Remington: The Science and Practice of Pharmacy (21^{st} Edition, University of the Sciences in Philadelphia, 2005). Ointments are semi-solid preparations that are typically based on petrolatum or other petroleum derivatives. The specific ointment base to be used, as will be appreciated by those skilled in the art, is one that will provide for optimum drug delivery, and, preferably, will provide for other desired characteristics as well, e.g., emolliency or the like. Creams are viscous liquids or semisolid emulsions, either oil-in-water or water-in-oil. Cream bases are water-washable, and contain an oil phase, an emulsifier and an aqueous phase. The oil phase, also called the “internal” phase, is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation is generally a nonionic, anionic, cationic or amphoteric surfactant. Gels are semisolid, suspension-type systems. Single-phase gels contain organic macromolecules (polymers) distributed substantially uniformly throughout the carrier liquid, which is typically aqueous, but also, preferably, contain an alcohol such as ethanol or isopropanol and, optionally, an oil. In order to prepare a uniform gel, dispersing agents such as alcohol or glycerin can be added, or the gelling agent can be dispersed by trituration, mechanical mixing or stirring, or combinations thereof. Lotions are preparations to be applied to the skin surface without friction, and are typically liquid or semiliquid preparations in which solid particles, including the active agent, are present in a water or alcohol base. Lotions are usually suspensions of finely divided solids and will typically contain suspending agents to produce better dispersions as well as compounds useful for localizing and holding the active agent in contact with the skin. Pastes are semisolid dosage forms in which the active agent is suspended in a suitable base. Depending on the nature of the base, pastes are divided between fatty pastes or those made from single-phase aqueous gels.

Various additives, known to those skilled in the art, may be included in the topical formulations. For example, solvents, including relatively small amounts of alcohol, may be used to solubilize certain drug substances. Other optional additives include opacifiers, antioxidants, fragrance, colorant, gelling agents, thickening agents, stabilizers, surfactants and the like. Other agents may also be added, such as antimicrobial agents, to prevent spoilage upon storage, i.e., to inhibit growth of microbes such as yeasts and molds. For those drugs having an unusually low rate of permeation through the skin or mucosal tissue, it may be desirable to include a permeation enhancer in the formulation. The formulation may also contain irritation-mitigating additives to minimize or eliminate the possibility of skin irritation or skin damage resulting from the drug, the enhancer, or other components of the dosage form. The formulations may also contain ether physiologically acceptable excipients or other minor additives, such as fragrances, dyes, emulsifiers, buffers, cooling agents (e.g. menthol), antibiotics, stabilizers or the like. In some instances, one component may serve more than one function.

The concentration of the active agent in a topical formulation can vary a great deal, and will depend on a variety of factors, including the disease or condition to be treated, the nature and activity of the active agent, the desired effect, possible adverse reactions, the ability and speed of the active agent to reach its intended target, and other factors within the particular knowledge of the patient and physician. The formulations will typically contain on the order of 0.1 wt % to 50 wt % active agent, preferably 0.1 wt % to 5 wt % active agent, optimally 5 wt % to 20 wt % active agent.

The pharmaceutical compositions of the present invention may be formulated as a depot formulation for administration via intramuscular or subcutaneous injection. Depot formulations are efficient, well-tolerated, sustained or delayed release compositions of the active ingredient that are therapeutically effective for a number of weeks, such as at least one week, at least two weeks, at least three weeks, at least four weeks, at least five weeks, or at least six weeks or more. In addition to the active agent, additional ingredients may be used in the depot formulations of the present invention including surfactants, solubilizers, emulsifiers, preservatives, isotonicity agents, dispersing agents, wetting agents, fillers, solvents, buffers, stabilizers, lubricants, and thickening agents. A combination of additional ingredients may also be used. The amount of the active ingredient in a depot formulation will depend upon the severity of the chronic lung disease being treated.

The compositions of the present invention may be presented in unit dosage form and may be prepared by any of the methods well known in the art of pharmacy. The term “unit dosage form” is taken to mean a single dose wherein all active and inactive ingredients are combined in a suitable system, such that the patient or person administering the drug to the patient can open a single container or package with the entire dose contained therein, and does not have to mix any components together from two or more containers or packages. Typical examples of unit dosage forms are tablets or capsules for oral administration. These examples of unit dosage forms are not intended to be limiting in any way, but merely to represent typical examples in the pharmacy arts of unit dosage forms.

The compositions of the present invention may also be presented as a kit, whereby two or more components, which may be active or inactive ingredients, carriers, diluents, and the like, are provided with instructions for preparation of the actual dosage form by the patient or person administering the drug to the patient. Such kits may be provided with all necessary materials and ingredients contained therein, or they may contain instructions for using or making materials or components that must be obtained independently by the patient or person administering the drug to the patient.

### Alpha-1 Antitrypsin Deficiency (AATD)

Chronic lung disease results from a wide variety of underlying causes. Two such causes are alpha-1 antitrypsin deficiency and emphysema resulting from alpha-1 antitrypsin deficiency. Alpha-1 antitrypsin (AAT) deficiency (AATD) is an autosomal codominant condition characterized by low circulating levels of AAT protein. People with AATD are at a high risk of developing emphysema at an early age (Kelly E., et al., Respir. Med., 2010, 104:763-772) and thus suffer from emphysema resulting from alpha-1 antitrypsin deficiency. These individuals also have a significant risk of liver disease and a lesser risk of panniculitis skin disease. AATD is the only proven genetic risk factor for the development of chronic obstructive pulmonary disease (COPD) and even heterozygote individuals with the MZ mutation, who smoke, are at increased risk of developing lung disease (Molloy K., et al., Am. J. Respir. Crit. Care Med., 2014, 189:419-427). The most common severe variant associated with lung, liver, and skin disease is the Z mutation, occurring in greater than 95% of individuals with severe AATD (Brantly, M., et al., Am. J. Med., 1988, 84: 13-31). The most typical manifestation of AATD is emphysema, which is typically panacinar and predominantly involves the lung bases (Parr, D. G., et al., Am. J. Respir. Crit. Care Med., 2004, 170:1172-1178). Emphysema resulting from AATD causes a loss of lung function and may also contribute to systemic inflammation, due to the lack of AAT anti-inflammatory effects (McCarthy, C., et al., Ann. Am. Thorac. Soc., 2016, Vol 13, Suppl. 4, pp S297-S304).

AATD is an inflammatory disorder, and the neutrophil plays a key role in these inflammatory processes. Acute lung injury resulting from microbial or chemical damage results in the recruitment and activation of neutrophils to clear pathogens from the tissue. There is a significantly higher presence of neutrophils in the lungs of individuals with AATD compared with healthy individuals. Hubbard and colleagues (Hubbard, R. C., et al., J. Clin. Invest., 1991, 88:891-897) demonstrated that there was not only an increased number of neutrophils in AATD bronchoalveolar lavage fluid, but also that the neutrophil chemotactic index was elevated. Increased neutrophil number and inflammatory signaling have been negatively correlated with lung function (Little, S. A., et al., Am. J. Med., 2004, 112:446-452; Singh, D., et al., Respiratory Res., 2010, 11:77). The significant neutrophil burden in the lungs of patients with AATD contributes to increased proteolytic activity and inflammation (Malerba, M., et al., Thorax, 2006, 61:129-133; Bergin, D. A., et al., J. Clin. Invest., 2010, 120:4236-4250). Unrestrained elastase concentrations, as is the case in AATD, can lead to excessive cleavage of immune molecules and extracellular matrix, as well as further recruitment of neutrophils (Travis, J., et al., Am. J. Med., 1988, 84:37-42; Kafienah, W., et al., Biochem. J., 1998, 330:897-902).

Because of the decreased serum concentrations of AAT, the lungs of Z homozygotes (Pi*ZZ), as well as individuals with null variants (Pi*Null), have little defense against NE and thus have an imbalance of NE and AAT. Unrestrained elastase concentration in the lung interstitial tissue of individuals with AATD results in damage to the lung and extracellular matrix, as well as further recruitment of neutrophils (Greene et al., 2016). Compound 1 is a potent inhibitor of neutrophil elastase as described above. Thus, it is useful in the treatment of AATD or emphysema resulting from AATD.

Emphysema is a condition in which the air sacs of the lungs are damaged and enlarged, causing breathlessness. In people with emphysema, the air sacs in the lungs (alveoli) are damaged. Over time, the inner walls of the air sacs weaken and rupture which creates larger air spaces instead of many small ones. This pathology reduces the surface area of the lungs and, in turn, the amount of oxygen that reaches the bloodstream. The main cause of emphysema is long-term exposure to airborne irritants including tobacco smoke, marijuana smoke, air pollution, chemical fumes and dusts, and asbestos. Cigarette smoking is by far the most significant cause (Anariba, D. E., 2018, emedicine.medscape.com/article/295686-medication). Loss of lung tissue is the pathological correlate for the progression of emphysema of any origin. The progression rate of emphysema is determined by change in lung density measured by computed tomography (CT) scan of whole lung. Early onset emphysema resulting from AATD is frequently overlooked (Tortorici, M. A., Br. J. Clin. Pharmacol., 2017, 83:2386-2307) and, when detected, is treated with supportive and augmentation therapy as described above.

### Therapeutic Administration and Doses

The terms “administration of” or “administering a” Compound 1 should be understood to mean providing (4S)-4-[4-cyano-2-(methylsulfonyl)phenyl]-3,6-dimethyl-2-oxo-1-[3-(trifluoromethyl)phenyl]-1,2,3,4-tetrahydropyrimidine-5-carbonitrile or a salt, solvate, a solvate of a salt, or a polymorph, to the individual in need of treatment in a form that can be introduced into that individual's body in a therapeutically useful form and a therapeutically effective amount, including, but not limited to, oral dosage forms, such as tablets, capsules, syrups, suspensions, and the like.

The terms “treat”, “treating” and “treatment” of alpha-1 antitrypsin deficiency (AATD) or emphysema resulting from AATD all refer to reducing the frequency of symptoms or signs of AATD or emphysema resulting from AATD (including eliminating them entirely), avoiding the occurrence of AATD or emphysema resulting from AATD and/or reducing the severity of symptoms or signs of AATD or emphysema resulting from AATD.

The term “therapeutically effective amount” refers to a sufficient quantity of Compound 1, in a suitable composition and in a suitable dosage form to treat the noted disease conditions. The “therapeutically effective amount” will vary depending on the compound, the severity of the AATD or emphysema resulting from AATD, and the age, weight, etc., of the patient to be treated.

The present methods for treatment of AATD or emphysema resulting from AATD require administration of Compound 1, or a pharmaceutical composition containing Compound 1, or a salt, solvate, a solvate of a salt, or a polymorph, to a patient in need of such treatment. The compound and/or pharmaceutical compositions are preferably administered orally. Various delivery systems are known, (e.g., encapsulation in liposomes, microparticles, microcapsules, capsules, etc.) which can be used to administer Compound 1 and/or composition.

The amount of Compound 1, a pharmaceutically acceptable salt, polymorph, solvate, or solvates of salts thereof, that will be effective in the treatment of AATD or emphysema resulting from AATD in a patient will depend on the specific nature of the disease, and can be determined by standard clinical techniques known in the art. In addition, in vitro or in vivo assays may optionally be employed to help identify optimal dosage ranges. The specific dose level for any particular individual will depend upon a variety of factors including the activity of the composition, the age, body weight, general physical and mental health, genetic factors, environmental influences, sex, diet, time of administration, route of administration, rate of excretion, and the severity of the condition being treated.

Preferably, the dosage forms are adapted to be administered to a patient one, two, three or more times a day. More preferably, a therapeutically effective amount is taken once per day. Alternatively, a dose may be taken every other day, every third day, every fourth day or once a week as may be appropriate for a particular dosage form. Dosing may be provided alone or in combination with other drugs and may continue as long as required for effective treatment of AATD or emphysema resulting from AATD.

Compound 1 may be administered in combination with one or more additional therapies. In one embodiment, Compound 1 may be administered with augmentation therapy with fractionated blood plasma or human AAT. Commercially available AAT preparations include Prolastin, which is also known as Prolastin-C®, Prolastina and Pulmolast (Grifols, Barcelona, Spain), Alfalastin (LFB, Courtaboeuf Cedex, France), Aralast® NP (Baxter, Lexington, Mass.), Zemaira® and Respreeza (CSL Behring, King of Prussia, Pa.), and Glassia® (Baxalta US, Inc., Lexington, Mass.).

In another embodiment, Compound 1 may be administered in combination with another therapeutic agent or agents that treat or ameliorate AATD or emphysema resulting from AATD. Such therapeutic agents, which are differentiated from AAT augmentation therapy, include, but are not limited to, AAT modulators, gene therapy, RNA-based therapies, leukocyte elastase inhibitors or recombinant AAT. Examples of AAT modulators, such as AAT stimulators, include a recombinant human AAT fusion protein (rhAAT-Fc) (INBRX-101; InhibRx, Inc., La Jolla, Calif.) and small molecule correctors of defective (e.g., misfolded) AAT protein (VX-814, VX-864, Vertex Pharmaceuticals, Boston, Mass.; ZF-874, Z Factor Ltd., Cambridge, United Kingdom). Examples of gene therapy, such as SERPINA1 gene editing, include CRISPR/Cas9 technology (Intellia Therapeutics, Inc., Cambridge, Mass.; Beam Therapeutics, Inc., Cambridge, Mass.; Editas Medicine, Inc., Cambridge, Mass.) and adenoassociated virus vector (AAV) therapies (LEX-01, LEXEO Therapeutics, New York, N.Y.; LGB-004, LogicBio Therapeutics, Lexington, Mass.; APB-101; ApicBio, Cambridge, Mass.). Examples of RNA-based therapies include an RNAi-based liver-targeted SERPINA1 gene blocker (ARO-AAT; Arrowhead Pharmaceuticals, Pasadena, Calif.); a triplex-forming peptide nucleic acid oligomer and DNA correction sequence encapsulated in a nanoparticle (Trucode Gene Repair, Inc., South San Francisco, Calif.); and a dicer-substrate siRNA (DsiRNA) that targets SERPINA1 mRNA (DCR-A1AT; Dicerna Pharmaceuticals, Inc., Lexington, Mass.). An example of a leukocyte elastase inhibitor is ionodelestat (POL-6014; Santhera Pharmaceuticals AG, Pratteln, Switzerland). An example of a recombinant AAT is OsrAAT (Healthgen Biotechnology Co. Ltd., Wuhan, Hubei, China).

Patients with AATD or emphysema resulting from AATD often suffer from co-morbid conditions (Stoller, J. K., Am. J. Respir. Crit. Care Med., 2012,185(3):246-59). In another embodiment, Compound 1 may be administered in combination with another therapeutic agent or agents that treat or ameliorate other such co-morbid diseases or conditions. AATD can predispose to other lung diseases (e.g., bronchiectasis), liver disease (e.g., chronic hepatitis, cirrhosis and hepatoma) and skin disease (i.e., panniculitis). Patients with the Pi**ZZ genetic variation are particularly susceptible to chronic hepatitis, cirrhosis and hepatocellular carcinoma. The Pi**ZZ variation is also associated with vasculitis (especially anticytoplasmic antibody-positive vasculitis such as Wegener's granulomatosis). Therapeutic agents for these co-morbid conditions or diseases are known to one skilled in the art.

Dosage ranges of Compound 1 for oral administration may be stated in terms of total amount of drug administered over a certain frequency of administration. A certain amount of active ingredient may be given one or more times a day as appropriate according to the factors described above. For example, doses may be taken once a day, twice a day, three times a day, four times a day, or more. Suitable dosages range from 0.1 mg to 100 mg, and preferably, from 1 mg to 40 mg, one or more times a day. Suitable dosages are typically 0.10 mg, 0.15 mg, 0.20 mg, 0.25 mg, 0.5 mg, 0.75 mg, 1 mg, 2 mg, 2.5 mg, 3 mg, 4 mg, 5 mg, 6 mg, 7 mg, 8 mg, 9 mg, 10 mg, 15 mg, 20 mg, 25 mg, 30 mg, 40 mg, 50 mg, or 100 mg one or more times per day. Preferably, a dose of 1 mg, 2 mg, 5 mg, 10 mg, 20 mg or 40 mg is administered once per day.

Alternatively, dosage ranges of Compound 1 for oral administration may be stated in terms of a weight-dependent dose. Suitable does are generally 0.001 mg to 5 mg of drug per kilogram body weight (mg/kg), one or more times a day. Suitable weight-dependent dosages are typically 0.001 mg/kg, 0.0015 mg/kg, 0.002 mg/kg, 0.0025 mg/kg, 0.005 mg/kg, 0.0075 mg/kg, 0.01 mg/kg, 0.02 mg/kg, 0.025 mg/kg, 0.03 mg/kg, 0.04 mg/kg, 0.05 mg/kg, 0.06 mg/kg, 0.07 mg/kg, 0.08 mg/kg, 0.09 mg/kg, 0.1 mg/kg, 0.15 mg/kg, 0.2 mg/kg, 0.25 mg/kg, 0.3 mg/kg, 0.4 mg/kg, 0.5 mg/kg, 1 mg/kg, 2 mg/kg, 3 mg/kg, 4 mg/kg or 5 mg/kg one or more times per day. Dosage ranges may be readily determined by methods known to the skilled artisan. The amount of active ingredient that may be, for instance, combined with carrier materials to produce a single dosage form will vary depending upon the patient treated and the particular mode of administration.

### Determination of Therapeutic Effectiveness

AATD is diagnosed by a variety of methods in individuals with symptomatic COPD, who are generally between 32 and 41 years old at the time of detection (American Thoracic Society Documents, Am. J. Respir. Crit. Care Med., 2003, 168:818-900). These patients are often smokers who present with a variety of chronic symptoms including productive cough, bronchitis, asthma, bronchiectasis, and wheezing. However, some current or previous smokers, or nonsmokers, present with none of these symptoms. Pulmonary function is most easily determined by spirometry. In patients with AATD, the forced expiratory volume in one second (FEV1) is reduced with a normal or reduced vital capacity (FVC). The reduced FEV1/FVC ratio (obstructive impairment) is primarily due to the loss of elastic recoil due to parenchymal disease (emphysema) with a concomitant dynamic collapse of otherwise normal airways (American Thoracic Society Documents, 2003).

Chapman and others have shown that the rate of FEV1 loss is slower in patients who receive augmentation therapy when compared with those who did not (Chapman, et al., COPD, 2009, 6:177-84). However, these changes in FEV1 take place slowly over many years even when the AATD-related lung disease is rapidly progressing (Chapman, et al., Lancet, 2015, 25:386(9991):360-8). A more practical method of measuring the efficacy of treatments for AATD or emphysema resulting from AATD is the measurement of lung density using computed tomography (CT) scan (Chapman, et al., 2015). Spiral CT scans may be conducted at total lung capacity (TLC) or functional residual capacity (FRC).

Given that these presenting pulmonary symptoms may be due to causes other than AATD, genetic tests are performed to confirm the presence of mutations in the SERPINA1 gene including the detection of S and Z alleles to establish an AATD-related diagnosis. These tests involve a variety of biochemical methods including nephelometric (light scattering) measurement of AAT concentration. When serum levels are low (i.e., <100 mg/dl) or when pedigree analysis is needed to clarify familial patterns, phenotyping by isoelectric focusing (IEF) is used. Genotyping can be performed by allele-specific amplification (currently for the S and Z alleles) or by extracting genomic DNA from circulating mononuclear cells or from mouth swabs for direct analysis. (Ferrarotti, I., et al., J. Chronic Obstr. Pulmon. Dis., 2016, http://dx.dol.org/10.1080/15412555.2016.1241760). The presence of rare null alleles can be inferred from genotyping but not from phenotyping by IEF because null alleles do not produce protein that can be identified by a band on the IEF. Many clinicians advocate simultaneously assessing AAT serum levels and genotyping, which is available through some commercial dried blood spot kits and also in a free, confidential home-testing kit (http://www.alpha-1foundation.org/alphas/?c¼02-Get-Tested). The method of the present invention is limited to treating pulmonary patients with AATD or with emphysema resulting from AATD.

Lung tissue destruction, in particular, the degradation of mature elastin, is observed in AATD patients and patients with emphysema resulting from AATD (Ferrarotti, I., et al., 2016). Desmosine and isodesmosine (DES/IDES) are two crosslinking amino acids which occur only in the mature elastin fiber. Mature elastin degradation results in the production of a variety of crosslinked elastin peptides containing desmosine (DES) and isodesmosine (IDES), collectively known as desmosines (DESs) being released into the circulation, urine, and sputum. DESs are rare tetrafunctional amino acid isoforms that only occur in mature human elastin (Ma, S., et al., Proc. Natl. Acad. Sci. USA, 2003, 100(22):12941-12943; Zanaboni, G., et al., J. Chromatogr. B. Biomed. Appl., 1996, 683(1):97-107). Levels of DES and IDES are higher in patients with destructive lung diseases vs. healthy subjects when measured in sputum, serum and urine samples using well-established analytic methods. Thus, these amino acids serve as biomarkers for elastin degradation in AATD and emphysema resulting from AATD (Ferrarotti, I., et al., 2016). For example, DES levels measured in urine and plasma reflected a patient's clinical status and could easily be associated with type Z AAT-deficient patients with clinically significant emphysema (Ferrarotti, I., et al., 2016). Ma, et al. reported measurements (in urine, plasma, and sputum) of DESs as markers of elastin degradation in both AATD patients and non-AATD-related COPD subjects (Ma, S., et al., Chest, 2007, 131(5):1363-1371; Ma, S., et al., J. Chromatogr. B. Analyt. Technol. Biomed. Life Sci., 2011, 879(21):1893-1898).

The efficacy of the methods and compositions of the present invention in the treatment of AATD and emphysema resulting from AATD can be evaluated in human clinical trials conducted under appropriate standards and ethical guidelines as set forth by the U.S. Food and Drug Administration (FDA) and other international agencies. After the general safety and pharmacokinetics of a drug is determined in Phase 1 clinical trials typically conducted in healthy volunteers, Phase 2 trials assessing the safety and efficacy of the drug in patients with the condition to be treated or target disease are conducted. Typically, such trials are double-blinded and controlled, and may be dose-ranging. Double-blinded and controlled Phase 3 studies gather more information about safety and attempt to prove effectiveness by studying the target population at specific dosages and, optionally, by using the drug in combination with other drugs.

The following examples are offered by way of illustration and not by way of limitation.

### EXAMPLES

### Example 1. Preparation of Tablets

Compound 1, (4S)-4-[4-cyano-2-(methylsulfonyl)phenyl]-3,6-dimethyl-2-oxo-1-[3-(trifluoromethyl)phenyl]-1,2,3,4-tetrahydropyrimidine-5-carbonitrile, may be formulated as a tablet for oral use. Manufacture of these tablets utilizes standard pharmaceutical process technologies. All of the inactive pharmaceutical ingredients in the examples below comply with requirements of United States Pharmacopeia (USP), The National Formulary (NF), the European Pharmacopeia (Ph. Eur.) and/or the Japanese Pharmacopeia (Ph. Jap.) as noted and are tested and released according to the monograph for each ingredient specified in the indicated standard. Batch sizes vary according to the amounts needed for a particular clinical purpose. The two examples below demonstrate the qualitative/quantitative composition of exemplary dosages and are for illustrative purposes. It is understood that additional dosage sizes and batch amounts are contemplated by the present invention.

Example 1a. Preparation of 0.5 mg tablets. The batch composition for 0.5 mg oral tablets is shown in Table 1.

TABLE 1

| | | | |
|---|---|---|---|
| | Reference to | Percent of | Amount |
| Composition | Quality Standard | Blend | (g) |

Intragranular

| | | | |
|---|---|---|---|
| Micronized Compound 1 | In house | 0.588% | 17.50 |
| Hydroxypropyl- | Ph. Eur., | 2.00% | 59.50 |
| cellulose 5 cP | USP/NF | | |
| Croscarmellose sodium | Ph. Eur., | 4.00% | 119.0 |
| | USP/NF, | | |
| | Ph. Jap. | | |
| Lactose monohydrate | Ph. Eur., NF | 92.41% | 2,749.3 |
| Purified water in bulk¹ | N.A. | N.A. | N.A. |

Extragranular

| | | | |
|---|---|---|---|
| Magnesium stearate | Ph. Eur., Ph. Jap. | 1.00% | 29.8 |

Film Coating

| | | | |
|---|---|---|---|
| White Lacquer | Opadry ^{™} white² | N.A. | 122.50 |
| Purified water in bulk¹ | N.A. | N.A. | N.A. |

| | |
|---|---|
| Total | 3,097.6 |

¹Purified water in bulk is used as solvent that is removed during the manufacturing process.
²Contains: Hypromellose 15 cP, Ph. Eur., NF, Ph. Jap.; Macrogol, Ph. Eur., USP, Ph. Jap.; Titanium dioxide, Ph. Eur., Directive 95/45/EC, USP, Ph. Jap.

Using the amounts specified in Table 1, micronized Compound 1, sodium croscarmellose and lactose monohydrate and are mixed in a fluidized bed granulator. A solution of hydroxypropylcellulose in water is added as the granulation liquid. After granulation, drying, milling and screening, extra-granular magnesium stearate is added. The final blend is compressed into tablets, which are tested for uniformity of mass, thickness and resistance to crushing. The tablets are coated with a solution of Opadry™ in water. The coated tablets are visually inspected for defects. Tablets with visible coating defects are rejected.

Example 1b. Preparation of 1 and 5 mg tablets. The batch composition for 1 mg and 5 mg oral tablets are shown in Tables 2 and 3, respectively.

TABLE 2

| | | | |
|---|---|---|---|
| | Reference to | Percent of | Amount |
| Component | Quality Standard | Blend | (g) |

Intra-granular

| | | | |
|---|---|---|---|
| Micronized | In-house | 1.19% | 40.4 |
| Compound 1 | | | |
| Lactose monohydrate | USP/NF, Ph. Eur., | 45.91% | 1,560.8 |
| | Ph. Jap | | |
| Hydroxypropyl- | USP/NF, Jap Ph. | 2.00% | 68.0 |
| cellulose | Eur., Ph. | | |
| Croscarmellose | Ph. Eur., NF, | 4.00% | 136.0 |
| sodium | Ph. Jap. | | |
| Purified water in bulk¹ | N.A. | N.A. | N.A. |

Extra-granular

| | | | |
|---|---|---|---|
| Microcrystalline | NF, Ph. Eur., | 45.91% | 1,560.8 |
| cellulose | Ph. Jap. | | |
| Magnesium stearate | NF, BP/Ph. Eur., | 1.00% | 34.0 |
| | Ph. Jap. | | |

Film Coating

| | | | |
|---|---|---|---|
| White lacquer | Opadry ^{™} II white² | N.A. | 140.0 |
| Purified water in bulk¹ | N.A. | N.A. | N.A. |

| | |
|---|---|
| Total | 3,400.0 |

¹Purified water in bulk is used as solvent that is removed during the manufacturing process.
²Contains: Polyvinyl alcohol, Ph. Eur., USP, FCC, Ph. Jap.; Macrogol, Ph. Eur., USP, FCC, JECFA, Ph. Jap.; Titanium dioxide, Ph. Eur., USP, FCC, Ph. Jap., Chp, GB; Talc, USP, FCC, Ph. Eur., Ph. Jap., JECFA.

TABLE 3

| | | | |
|---|---|---|---|
| | Reference to | Percent of | Amount |
| Component | Quality Standard | Blend | (g) |

Intra-granular

| | | | |
|---|---|---|---|
| Micronized | In-house | 5.96% | 60.8 |
| Compound 1 | | | |
| Lactose monohydrate | USP/NF, Ph. Eur., | 43.52% | 443.9 |
| | Ph. Jap | | |
| Hydroxypropyl- | USP/NF, Ph. Eur., | 2.00% | 20.4 |
| cellulose | Ph. Jap | | |
| Croscarmel lose | Ph. Eur., NF, | 4.00% | 40.8 |
| sodium | Ph. Jap. | | |
| Purified water in bulk¹ | N.A. | N.A. | N.A. |

Extra-granular

| | | | |
|---|---|---|---|
| Microcrystalline | NF, Ph. Eur., | 43.52% | 443.9 |
| cellulose | Ph. Jap. | | |
| Magnesium stearate | NF, BP/Ph. Eur., | 1.00% | 10.2 |
| | Ph. Jap. | | |

Film Coating

| | | | |
|---|---|---|---|
| White lacquer | Opadry ^{™} II white² | N.A. | 42.0 |
| Purified water in bulk¹ | N.A. | N.A. | N.A. |

| | |
|---|---|
| Total | 1,062.0 |

¹Purified water in bulk is used as solvent that is removed during the manufacturing process.
²Contains: Polyvinyl alcohol, Ph. Eur., USP, FCC, Ph. Jap.; Macrogol, Ph. Eur., USP, FCC, JECFA, Ph. Jap.; Titanium dioxide, Ph. Eur., USP, FCC, Ph. Jap., Chp, GB; Talc, USP, FCC, Ph. Eur., Ph. Jap., JECFA.

Using the amounts specified in Tables 2 and 3, micronized Compound 1, sodium croscarmellose and lactose monohydrate and are mixed in a high shear granulator. A solution of hydroxypropylcellulose in water is added as the granulation liquid. After granulation, drying, milling and screening, extra-granular microcrystalline cellulose and magnesium stearate are added, with blend uniformity being tested prior to addition of the magnesium stearate. The final blend is compressed into tablets, which are tested for uniformity of mass, thickness and resistance to crushing. The tablets are coated with a solution of Opadry™ II in water. The coated tablets are visually inspected for defects. Tablets with visible coating defects are rejected.

### Example 2. Phase 1 Clinical Study of Compound 1 in Healthy Patients

Study Description. A Phase 1, single-center, randomized, double-blind, placebo-controlled single-ascending dose study designed to evaluate the safety, tolerability, and pharmacokinetics (PK) of Compound 1 in healthy subjects was conducted in accordance with Good Clinical Practice (GCP), the ethical principles that have their origin in the Declaration of Helsinki, and all other applicable laws, rules and regulations.

Within each dose cohort, subjects were randomized in a 3:1 ratio (6 active and 2 placebo) to receive either Compound 1 or placebo. Following Screening, subjects received single doses of study drug and were monitored during an in-clinic period and an out-patient follow-up period. Subjects were confined to the study site for Study Days −2 through 7 to collect PK and safety assessments. Following discharge from the study site on Study Day 7, subjects returned to the study site on Study Days 14, 21, 28, and 35.

Results. A total of 36 subjects received Compound 1 (at doses in the range 1 to 40 mg) and 12 subjects received placebo. Of the original 48 subjects randomized, three discontinued for administrative reasons. A Dose Escalation Review Committee assessed all available safety and PK data from each cohort and agreed that dose escalation was appropriate in each case (up to the planned maximum dose of 40 mg).

The overall distribution of treatment-emergent adverse events was comparable in each of the treatment groups with 29% (14 of 48) subjects experiencing one or more adverse events (AEs). There were no notable differences in the occurrence of AE by body system and no clear relationship of dose. The AE rate for Compound 1 subjects was somewhat lower than that in the control (placebo) subjects. Headache which was experienced by more subjects than any other AE, was observed in only one Compound 1 subject. There were no serious AEs and no discontinuations or deaths. There were no clear effects of Compound 1 on laboratory safety evaluations (clinical chemistry and hematology).

The PK of Compound 1 appeared to be well behaved with proportional increases in exposure (AUC and Cmax) with dose. The observed PK would support once daily administration of Compound 1.

### Example 3. Phase 2 Clinical Study of Compound 1 in Patients with AATD

Study Description. This study is a Phase 2, multicenter, double-blind, randomized (1:1), placebo-controlled, proof-of-concept study to evaluate the safety and tolerability, as well as the effect on pharmacodynamic markers, of Compound 1 administered daily for 12 weeks, in patients with confirmed AATD (Alpha-1 ZZ genotype [Pi*ZZ]) or Alpha-1 Null phenotype [Pi*Null phenotype], AAT levels <11 μM (0.5 g/L)), and AATD-related emphysema. The trial is conducted in accordance with Good Clinical Practice (GCP), the ethical principles that have their origin in the Declaration of Helsinki, and all other applicable laws, rules and regulations. Eligible patients will be enrolled and randomized within 30 days of screening in a 1:1 ratio (1 active and 1 placebo), to receive Compound 1 20 mg daily or 10 mg daily or matching placebo daily for 84 days (12 weeks). Compound 1 will be provided as immediate release (IR) 5-mg tablets.

Participants will be screened to yield approximately 60 enrolled study participants. Patients will take oral doses of Compound 1 20 mg QD (four 5-mg tablets) or Compound 1 10 mg (two 5-mg tablets plus two placebo tablets) or placebo QD (four placebo tablets) for 84 days (12 weeks) on an outpatient basis. The study drug will be taken daily, orally with water, ideally in the morning at approximately the same time each day. Study drugs may be taken either fasting or with food. Grapefruit and grapefruit juice should be avoided. Each subject will be asked to attend a follow-up visit on Study Day 106.

Baseline procedures will include vital signs, abbreviated medical history, abbreviated physical examination, hematological and biochemical analysis, serum pregnancy test for females and blood draws for eligibility. Post-bronchodilators spirometry (FEV, and forced vital capacity [FVC]), and an ECG, will also be done at baseline. In addition, chest X-ray and lung density as assessed by spiral computerized tomography (CT) scans at total lung capacity (TLC) and fundamental residual capacity (FRC) may be performed. At the baseline visit, enrolled patients will be dispensed study drug and a daily diary. Blood samples will be drawn periodically to measure Compound 1 levels.

The interpretation of safety and tolerability, as applicable, will be assessed based on the collection of all available safety data, including adverse events/serious adverse events, physical examination findings, clinical laboratory parameters, vital signs, and ECGs.

Statistical Methods. Demographic and baseline characteristics, such as age, sex, race/ethnicity, and baseline PROs, using e.g., EQ-5D and CAT, will be summarized by treatment arm in all randomized participants. The PD (e.g., biomarker levels) response to dosing with Compound 1 20 mg QD or Compound 1 10 mg QD will be compared to placebo to be evaluated, as will all efficacy endpoints. In brief, efficacy and exploratory endpoints will be compared between active and placebo arms at day 8, day 15, day 29, day 57, day 84, and day 106 compared to baseline (day 1), adjusting for covariates including concomitant steroid use, as required. Data will be analyzed according to the Statistical Analysis Plan.

Safety Analysis. Safety data, including AEs, vital signs, physical examination results, and clinical laboratory evaluations, will be summarized. Descriptive statistics will be provided, where appropriate.

Pharmacokinetics. Plasma PHP-303 levels will be measured in each treatment group at multiple time points. Samples will be collected pre-dose, 15 minutes, 30 minutes, and 4 hours after dosing on day 1, pre-dose of day 8, day 15, day 29, day 57, day 84, and on day 106. Plasma concentrations will be summarized by nominal day and time of collection. No formal PK parameters (e.g. Cₘₐₓ or AUC) will be reported. Missing data will not be imputed. Plasma concentrations will be summarized for Compound 1 at day 1, day 8, day 15, day 29, day 57, day 84, and day 106. In addition, sputum concentrations may be summarized for Compound 1 at day 1, day 57, and day 84 (induced sputum when available) and for Compound 1 at day 8, day 15, day 29, and day 106 (spontaneous sputum when available).

Pharmacodynamics. Pre- and post-treatment levels of biomarkers related to target NE engagement will be analyzed using descriptive statistics. Possible analyses include the following parameters: blood NE activity; bronchoalveolar lavage NE activity; blood desmosine/isodesmosine levels; urine desmosine/isodesmosine levels, and induced sputum.

Additional clinical trials with an appropriate design for the stage of clinical development may be conducted to test the efficacy of Compound 1 in the treatment of AATD patients. Further trials utilizing different dosage levels of the active ingredient or to differentiate between optimal doses or dosing schedules may be conducted. Further, the efficacy of the drug in specific populations, such as the elderly with AATD, children with AATD, or AATD patients with common co-morbidities or other pathological conditions may be determined in additional clinical trials conducted in a similar fashion. In particular, patients with the Pi*ZZ genetic variant having hepatic dysfunction, including hepatitis, cirrhosis, and hepatocellular carcinoma, will need to be included in further clinical trials.

All publications and patent applications mentioned in this specification are herein incorporated by reference to the same extent as if each individual publication or patent application were specifically and individually indicated to be incorporated by reference.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A method of treating chronic lung disease, comprising administering a therapeutically effective amount of (4S)-4-[4-cyano-2-(methylsulfonyl)phenyl]-3,6-dimethyl-2-oxo-1-[3-(trifluoromethyl)phenyl]-1,2,3,4-tetrahydropyrimidine-5-carbonitrile or a pharmaceutically acceptable salt, polymorph, solvate, or solvates of the salts thereof to a patient in need of treatment, wherein the therapeutically effective amount comprises a dosage of 1 mg, 2 mg, 5 mg, 10 mg, 20 mg or 40 mg once a day, and wherein the chronic lung disease is selected from the group consisting of alpha-1 antitrypsin deficiency or emphysema resulting from alpha-1 antitrypsin deficiency.

2. The method of claim 1, wherein the chronic lung disease is alpha-1 antitrypsin deficiency.

3. The method of claim 1, wherein the chronic lung disease is emphysema resulting from alpha-1 antitrypsin deficiency.

4. The method of claim 1, further comprising administering one or more additional therapies.

5. The method of claim 4, wherein the additional therapy is augmentation therapy with human alpha-1 antitrypsin.

6. The method of claim 4, wherein the additional therapy is treatment with a therapeutic agent when administered to a patient by itself treats or ameliorates alpha-1 antitrypsin deficiency or emphysema resulting from alpha-1 antitrypsin deficiency.

7. The method of claim 6, wherein the therapeutic agent is an alpha-1 antitrypsin modulator, gene therapy, RNA-based therapy, a leukocyte elastase inhibitor or recombinant AAT.

8. A pharmaceutical composition for the treatment of alpha-1 antitrypsin deficiency or emphysema resulting from alpha-1 antitrypsin deficiency comprising (4S)-4-[4-cyano-2-(methyl sulfonyl)phenyl]-3,6-dimethyl-2-oxo-1-[3-(trifluoromethyl)phenyl]-1,2,3,4-tetrahydropyrimidine-5-carbonitrile or a pharmaceutically acceptable salt, polymorph, solvate, or solvates of the salts thereof and a pharmaceutically acceptable carrier.

9. The pharmaceutical composition of claim 8, wherein the pharmaceutical composition is formulated as a tablet.

10. The pharmaceutical composition of claim 9, wherein the tablet comprises one or more diluents, disintegrants, surfactants or lubricants.

11. The pharmaceutical composition of claim 8, wherein the pharmaceutical composition comprises 1 mg, 2 mg, 5 mg, 10 mg, 20 mg or 40 mg of (4S)-4-[4-cyano-2-(methylsulfonyl)phenyl]-3,6-dimethyl-2-oxo-1-[3-(trifluoromethyl)phenyl]-1,2,3,4-tetrahydropyrimidine-5-carbonitrile or a pharmaceutically acceptable salt, polymorph, solvate, or solvates of the salts thereof.

12. A method of treating alpha-1 antitrypsin deficiency or emphysema resulting from alpha-1 antitrypsin deficiency in a patient in need of such treatment comprising administering to said patient a therapeutically effective amount of a pharmaceutical composition according to claim 8.

13. The method of claim 12, wherein the pharmaceutical composition comprises 1 mg, 2 mg, 5 mg, 10 mg, 20 mg or 40 mg of (4S)-4-[4-cyano-2-(methylsulfonyl)phenyl]-3,6-dimethyl-2-oxo-1-[3-(trifluoromethyl)phenyl]-1,2,3,4-tetrahydropyrimidine-5-carbonitrile or a pharmaceutically acceptable salt, polymorph, solvate, or solvates of the salts thereof.

14-20. (canceled)
